(19) United States
(12) Patent Application Publication
(10) Pub. No.: US 2012/0122172 A1
(43) Pub. Date: May 17, 2012

(54) METHOD FOR PRODUCING ETHANOL FROM XYLOSE USING RECOMBINANT SACCHAROMYCES CEREVISIAE TRANSFORMED TO ELIMINATE FUNCTIONS OF GENES INVOLVED IN TOR SIGNAL TRANSDUCTION PATHWAY

(75) Inventors: Jin-Ho Seo, Seoul (KR); Yong-Cheol Park, Seoul (KR)

(73) Assignee: SNU R&DB FOUNDATION, Seoul (KR)

(21) Appl. No.: 13/216,383

(22) Filed: Aug. 24, 2011

Related U.S. Application Data
(63) Continuation of application No. PCT/KR2010/008078, filed on Nov. 16, 2010.

(51) Int. Cl. C12P 7/06

(52) U.S. Cl. 435/161

(57) ABSTRACT

Disclosed is a method for producing ethanol from zylose using a Saccharomyces cerevisiae strain transformed to eliminate functions of genes involved in TOR signal transduction pathways. The method provides an increase in ethanol yield and production efficiency, as compared to a control group, and enables production of ethanol at higher yield and high production efficiency by further eliminating acetaldehyde dehydrogenase which mediates production of acetic acid (by-product).

### CROSS-REFERENCE TO RELATED APPLICATION

This is a continuation application of PCT/KR2010/008078 filed on Nov. 16, 2010, the entire contents of which are incorporated herein by reference.

### TECHNICAL FIELD

The present invention relates to a method for producing ethanol from xylose using recombinant Saccharomyces cerevisiae. More particularly, the present invention relates to a method for producing ethanol from xylose using recombinant Saccharomyces cerevisiae wherein functions of genes involved in TOR signal transduction pathways have been knocked out of the recombinant Saccharomyces cerevisiae.

### BACKGROUND ART

At present, mankind faces a serious problem of exhaustion of natural resources. Exhaustion of natural resources together with environmental pollution is a threat to civilization. Among resources, exhaustion of oil is particularly serious. Exhaustion of oil has been predicted for several decades. In accordance with some reports, oil will be entirely exhausted after 100 to 150 years, in spite of considerable advances in drilling technology.

Accordingly, development of alternative energy using solar power, wind power or nuclear power is required and a great deal of attempt to commercialize alternative energy has been made. Nuclear power as well as oil is emerging as a main energy source essential for earth and importance of energy such as solar power or wind power is gradually also extended.

However, oil is still of great importance in the field of energy for transportation, since alternative energy such as nuclear power focuses only on power generation. Accordingly, there is an increasing demand for development of materials for transportation which can replace oil. At present, ethanol is attracting considerable attention as an oil alternative and has already been put to use as an alternative energy source in Brazil and the U.S.

Ethanol, the main ingredient in alcoholic beverages, has been consumed since the development of alcoholic beverages. Meanwhile, in the conventional low-cost oil age, the cost of manufacturing ethanol is higher than the price of oil, and thus ethanol is not cost-competitive. However, it has been reported that ethanol is becoming increasingly cost-competitive and will eventually overtake the price competitiveness of oil, taking into consideration gradual exhaustion of oil deposits and increased oil prices.

Ethanol, used as a transportation fuel, is prepared from sugarcane, corn, etc. Sugarcane is a raw material of sugar and corn is a raw material of foods. For this reason, the use of these resources for the preparation of ethanol causes, as a side-effect, an increase in sugar or corn prices and, as an ethical problem, use of the grain for fuel, rather than food.

Accordingly, a great deal of research has focused on the development of alternatives to sugarcane and corn. Xylose, found at high concentrations in waste wood materials or forestry by-products, is one potential candidate.

Xylose is a renewable resource, which is recovered from wood byproducts generated in the process of producing pulp, etc. Use of xylose does not entail a price increase of alternative materials and is free from ethical problems and a great deal of research associated therewith is thus underway at present.

Meanwhile, Saccharomyces cerevisiae is well-known as a strain for producing ethanol in the preparation of fermented liquor such as coarse liquor. Saccharomyces cerevisiae is actively utilized as a host for the preparation of useful medicines and its use as a host for preparing ethanol is actively researched. However, considering metabolism of xylose, wild-type Saccharomyces cerevisiae has neither xylose reductase (XR) nor xylitol dehydrogenase (XDH) and thus, disadvantageously, cannot metabolize xylose.

Accordingly, in order to allow Saccharomyces cerevisiae to metabolize xylose, a great deal of research is being conducted into incorporation of these enzymes into Saccharomyces cerevisiae. From research results, it can be confirmed that xylose is substantially metabolized. In addition, when Saccharomyces cerevisiae metabolizes xylose, ethanol is produced as a byproduct. Accordingly, some researchers are interested in xylose as a carbon source to produce ethanol which comes into the spotlight as an alternative energy source.

In this regard, preparation of ethanol from xylose causes problems of low preparation yield and low production efficiency. An attempt to overcome low yield and production efficiency via increase of absorption of xylose in strains and control of rate determining steps of metabolic pathways has been made. However, higher preparation yield and production efficiency are required in the art.

The above information disclosed in this Background section is only for enhancement of understanding of the background of the invention and therefore it may contain information that does not form the prior art that is already known in this country to a person of ordinary skill in the art.

### SUMMARY OF THE DISCLOSURE

When ethanol is produced from xylose using Saccharomyces cerevisiae, an increase in preparation yield and low production efficiency has been limited only using conventionally actively researched metabolic engineering to overcome the drawback associated with rate determining step.

The inventors of the present invention noted variation of gene expression behaviors caused by variation in signal transduction in cells due to the limitation during fermentation of ethanol from xylose and deleted intermediate genes of the Tor signal transduction pathway which are operated in nutrient deficiency in order to improve ethanol yield and production efficiency.

In addition, the present inventors deleted genes involved in re-absorption of produced ethanol in order to further improve ethanol yield and production efficiency.

In accordance with the present invention, the above and other objects can be accomplished by the provision of a method for preparing ethanol from xylose using recombinant Saccharomyces cerevisiae wherein the recombinant Saccharomyces cerevisiae is transformed to express xylose reductase (XR) and xylitol dehydrogenase; (XDH), wherein the recombinant Saccharomyces cerevisiae is transformed to over-express xylulokinase (XK), wherein genes involved in TOR signal transduction pathways in the recombinant Saccharomyces cerevisiae are partially disrupted or entirely deleted to eliminate functions of the genes.

Saccharomyces cerevisiae is commercially available as an ethanol-producing strain, but does not utilize xylose as a sole carbon source. This is because Saccharomyces cerevisiae has neither xylose reductase (XR) nor xylitol dehydrogenase (XDH), thus having no metabolic activity to convert xylose into xylulose.

Accordingly, XR and XDH enzymes should be integrated into hosts in order to enable production of ethanol. In the Saccharomyces cerevisiae of the present invention wherein XR and XDH are integrated and thus transformed, xylose is converted into xylulose, the xylulose is converted into xylulose 5-phosphate through additionally integrated xylulokinase (XK), and metabolism is performed via the pentose phosphate cycle. XK is an enzyme present in yeasts, can produce ethanol from xylose when not over-expressed and only XR and XDH are transformed into the strain. In this case, yield and production efficiency are disadvantageously considerably low. In order to overcome this drawback, over expression of XK is preferable (See FIG. 1).

Meanwhile, the present invention is characterized in that genes involved in TOR signal transduction are partially disrupted or entirely deleted from the Saccharomyces cerevisiae strain which is transformed as mentioned above and can thus produce ethanol, to eliminate functions of the genes.

The TOR signal transduction pathway is a nutrient-starvation signaling pathway, which operates when glucose lacks outside of strains as well as when xylose is used as a carbon source to produce ethanol. In the present invention, genes involved in TOR signal transduction are partially disrupted or entirely deleted to eliminate functions thereof and thereby prevent normal operation of the TOR signal transduction pathway, and make Saccharomyces cerevisiae to be fermented in glucose. It can be confirmed from the following tests that removal of functions of genes involved in TOR signal transduction pathway causes an increase in yield and production efficiency of ethanol.

Partially disruption of genes as one means of removal of gene functions may be carried out in accordance with homologous recombination, as illustrated in FIG. 2, and entire deletion of genes may be carried out in accordance with double homologous recombination, as illustrated in FIG. 3.

Meanwhile, the gene involved in TOR signal transduction, the function of which is to be eliminated, is selected from PPH21, PPH22, PPH3, PPM1, TOR1, TPD3 and MAF1 (Reference: Saccharomyces Genome Database, http://www.yeastgenome.org/)/

Meanwhile, generally, xylose reductase (XR) used in the present invention utilizes NADPH as a cofactor and XDH utilizes NAD⁺ as a cofactor. When NADH-dependent XR is used, rather than NADPH-dependent XR, NADH and NAD⁺ are coupled with each other between XR and XDH, thus solving the problem, namely, deterioration in production efficiency, which is caused by limited cofactor supply.

Accordingly, in the present invention, preferred is use of xylose reductase (XR) which utilizes NADH as a cofactor.

Meanwhile, preferred is recombinant Saccharomyces cerevisiae wherein acetaldehyde dehydrogenase-encoding genes which convert acetaldehyde into acetic acid are partially disrupted or entirely deleted and thus lose their function. Thus, production of acetic acid as a by-product is prevented and ethanol can thus be produced at high yield and high production efficiency. For example, the acetaldehyde dehydrogenase-encoding gene may be ALD6.

Meanwhile, gene names are written in italics and protein names are not written in italics.

As apparent from the above description, the present invention provides a method for producing ethanol using Saccharomyces cerevisiae, wherein yield and production efficiency of ethanol can be increased using a Saccharomyces cerevisiae strain which is transformed to eliminate functions of genes involved in TOR signal transduction pathways, and ethanol can be produced at a higher yield and production efficiency by removing acetaldehyde dehydrogenase which mediates production of acetic acid (by-product).

### BRIEF DESCRIPTION OF DRAWINGS

The above and other objects, features and other advantages of the present invention will be more clearly understood from the following detailed description taken in conjunction with the accompanying drawings, in which:

FIG. 1 is a flowchart illustrating a process for producing ethanol from xylose according to the present invention;

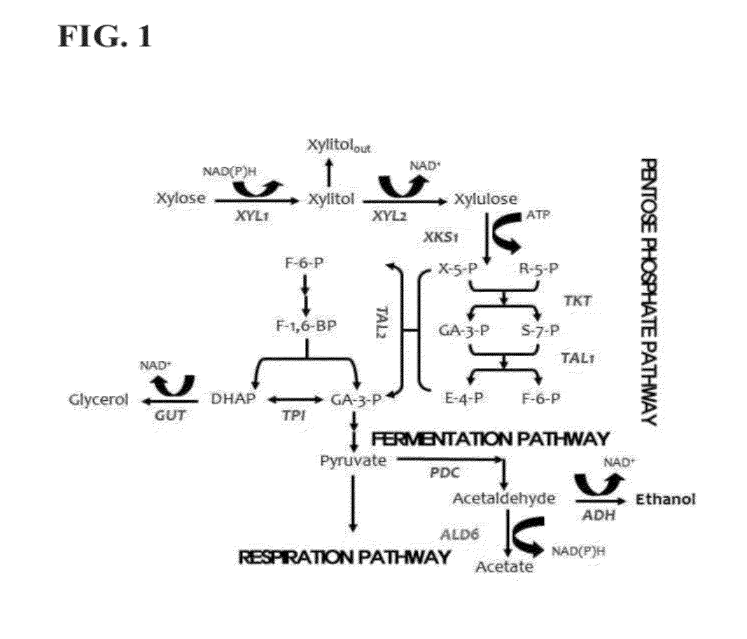

FIG. 2 is a schematic view illustrating a process wherein target genes are disrupted by homologous recombination;

FIG. 3 is a schematic view illustrating a process wherein target genes are deleted by double homologous recombination;

FIG. 4 shows fermentation results of SX3 strains, SX3::Δpph21 strains, SX3::Δpph22 strains and SX3::Δpph3 strains; and

FIG. 5 shows fermentation results of SX3 strains, SX3::Δpph21 strains, SX5::Δald6 strains and SX3::Δpph21::Δald6 strains.

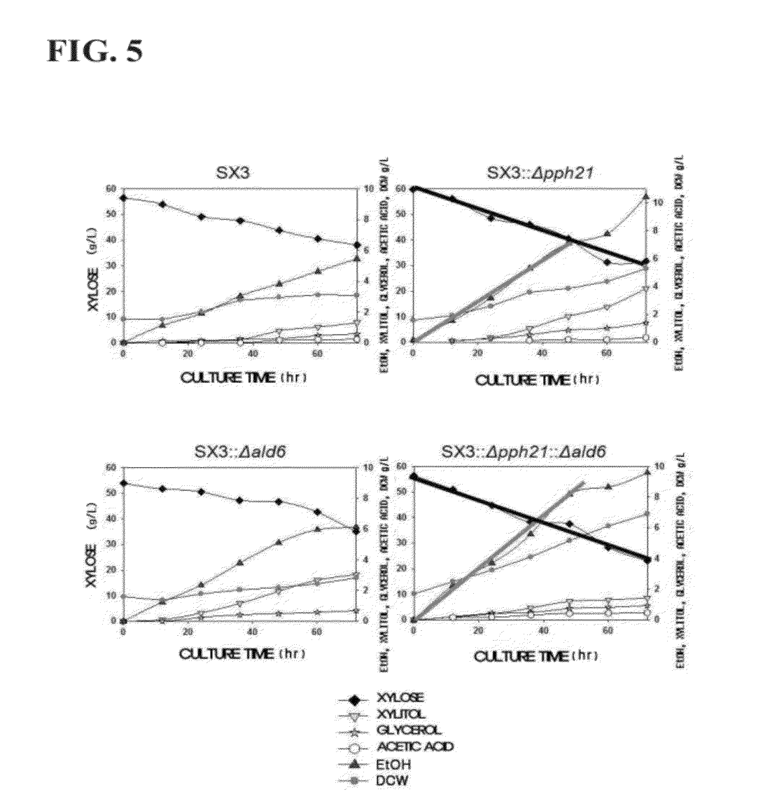

### DETAILED DESCRIPTION OF THE DISCLOSURE

Hereinafter, the following examples and experimental examples will be provided for a further understanding of the invention. The following examples are for illustrative purposes only and are not intended to limit the scope of the present invention.

### Example 1

### Production of Transformed Saccharomyces Cerevisiae Strains

In this example, recombinant Saccharomyces cerevisiae to be used in the following Examples was prepared.

Meanwhile, gene recombination and methods for producing transformation systems are not described in detail in this example since they are well-known in the art of genetic engineering.

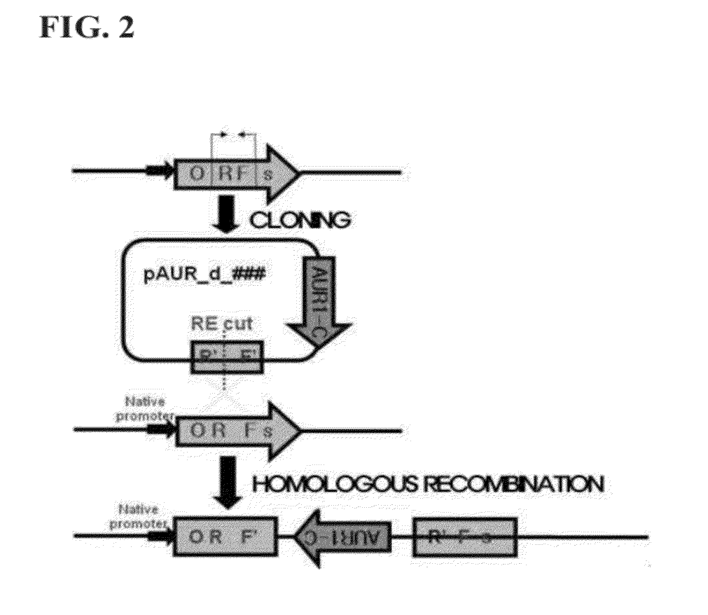

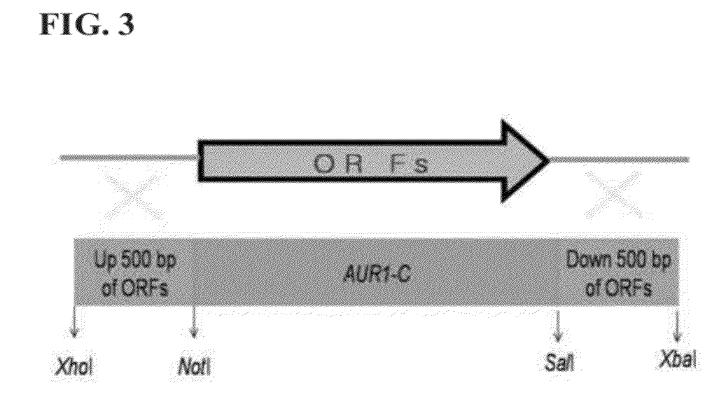

Meanwhile, functions of PPH21, PPH22, PPH3 and ALD6 were eliminated by homologous recombination, as shown in FIG. 2 (Burke, Dawson et al., Methods in yeast genetics, Cold Spring Harbor Laboratory Press New York. 2000) and functions of PPM1, TOR1, TPD3 and MAF1 were eliminated by double homologous recombination, as shown in FIG. 3 (Burke, Dawson et al., Methods in yeast genetics, Cold Spring Harbor Laboratory Press New York. 2000).

In the case of gene disruption as disclosed in FIG. 2, genes (ORFs) to be disrupted are recombined in chromosomes in two forms of ORF′ and R′Fs by homologous recombination. At this time, ORFs are not completely formed and none of the two forms are expressed in ORFs. Finally, functions of ORFs were eliminated in strains.

In the case of gene deletion as disclosed in FIG. 3, up 500 bp and down 500 bp of genes (ORFs) to be deleted were cloned, a marker (AUR1-C) was interposed therebetween to produce nucleic acid fragments and the nucleic acid fragments were inserted into strains to induce homologous recombination in up and down regions of ORFs. As a result, the ORFs were deleted and the marker replaces the ORFs.

The Saccharomyces cerevisiae D452-2 used herein as a host was obtained from professor Makino of Kyoto University in Japan (Seiya Watanabe, Ahmed Abu Saleh, Seung Pil Pack, Narayana Annaluru, Tsutomu Kodaki and Keisuke Makino, 2007, Ethanol production from xylose using recombinant Saccharomyces cerevisiae expressing protein-engineered NADH-preferring xylose reductase from Pichia stipitis. Microbiol. 153:3044-3054).

Vectors, YEpM4XR(WT), YEpM4XR(R276H) and pPGKXDH(WT) were also collected from professor Makino of Kyoto University in Japan and the XR(R276H) enzyme mutated by subjecting wild-type XR to point mutation exhibits higher selective affinity for NADH than NADPH, as compared to wild-type XR (See FIG. 2, Seiya Watanabe, Ahmed Abu Saleh, Seung Pil Pack, Narayana Annaluru, Tsutomu Kodaki and Keisuke Makino, 2007, Ethanol production from xylose by recombinant Saccharomyces cerevisiae expressing protein-engineered NADH-preferring xylose reductase from Pichia stipitis Microbiol. 153:3044-3054).

The parent vectors used for the production of YIpXR^{WT}-XDH^{WT} and YEpM4XR (R276H), YIp5 and delta ISXK, were collected from Tae-Hee Lee, the previous researcher, the Graduate School of Seoul National University (Tae-Hee Lee, Metabolic engineering studies on production of ethanol from xylose by recombinant Saccharomyces cerevisiae, M.S. Thesis, Seoul National University, 2000).

pAUR101 used for gene disruption and pET-26b(+) used for gene deletion were commercially obtainable from Takara, Japan, and PPH21, PPH22, PPH3, PPM1, TOR1, TPD3, MAF1 and ALD6 genes were obtained by cloning S. cerevisiae CEN.PK2-1D.

The following Table 1 shows strains and gene types thereof produced in this example.

TABLE 1
Strains prepared in this example

| Strains | Gene type | Plasmids used |
|---|---|---|
| D452-2 | Matα, leu2 his3 ura3 can1 | |
| D452-2/ | D452-2, ura3::URA3, leu2::LEU2 | YEpM4XR(WT), |
| pXRWT/pXDH | YEpM4XR(WT), pPGKXDH(WT) | pPGKXDH(WT) |
| D452-2/ | D452-2, ura3::URA3, leu2::LEU2, | YEpM4XR(R276H), |
| pXR^{MUT}/pXDH | YEpM4XR(R276H), pPGKXDH(WT) | pPGKXDH(WT) |
| D452-2/ | D452-2, ura3::URA3 | YIpXR^{WT}-XDH^{WT} |
| YIpXR^{WT}-XDH | P_{PGK}-XYL1^{WT}-T_{PGK}, P_{PGK}-XYL2^{WT}-T_{PGK} | |
| SX2 | D452-2, ura3::URA3 | YIpXR^{R276H}-XDH^{WT} |
| | P_{PGK}-XYL1^{MUT}-T_{PGK}, P_{PGK}-XYL2^{WT}-T_{PGK} | |
| SX3 | SX2, | delta ISXK |
| | Ty1-delta::P_{GPD}-XKS1-T_{GPD}-neo^{r} | |
| SX3::Δppm1 | SX3, PPM1::AUR1_C | pET-26b(+) |
| SX3::Δtor1 | SX3, TOR1::AUR1_C | |
| SX3::Δtpd3 | SX3, TPD3::AUR1_C | |
| SX3::Δmaf1 | SX3, MAF1::AUR1_C | |
| SX3::Δpph21 | SX3, PPH21::pAUR_d_PPH21 | pAUR101 |
| SX3::Δpph22 | SX3, PPH22::pAUR_d_PPH22 | |
| SX3::Δpph3 | SX3, pph3::pAUR_d_PPH3 | |
| SX3::Δald6 | SX3, ALD6::pAUR_d_ALD6 | |
| SX3::Δpph21:: | SX3::Δpph21, | |
| Δald6 | ALD6::p425_d_ALD6 | |

1) WT means wild type.
2) MT means mutant type.

SX2 is a strain wherein XR^{mut} and XDH are integrated in chromosomes by homologous recombination and SX3 is a strain wherein XK is further integrated in the delta sequence of chromosomes to provide higher ethanol production efficiency than SX2.

“SX3::Δppm1” is a strain obtained by removing PPM1 genes from a SX3 strain, “SX3::Δtor1” is a strain obtained by removing TOR1 genes from SX3 strains, “SX3::Δtpd3” is a strain obtained by removing TPD3 genes from SX3 strain, “SX3::Δmaf1” is a strain obtained by removing MAF1 genes from SX3 strains, “SX3::Δpph21” is a strain obtained by removing PPH21 genes from SX3, “SX3::Δpph22” is a strain obtained by removing PPH22 genes from SX3, “SX3::Δpph3” is a strain obtained by removing PPH3 genes from SX3 and “SX3::Δald6” is a strain obtained by removing ALD6 genes from SX3. “SX3::Δpph21::Δald6” is a strain obtained by removing PPH21 and ALD6 genes from SX3.

### Example 2

### Fermentation of Ethanol Using SX3, SX3::Δpph21, SX3::Δpph22 and SX3::Δpph3 Strains Among Strains Produced in Example 1

Fermentation of ethanol was performed using SX3, SX3::Δpph21, SX3::Δpph22 and SX3::Δpph3 strains among strains produced in Example 1.

A multifermentation bath (KF-1L, manufactured by Kobiotech Co., Ltd.) with a size of 1 L was used for fermentation and operation volume was 500 mL. The fermentation bath was maintained at a temperature of 30° C. and a fermentation solution was maintained at a pH of 5.5.

Stirring was performed at a rate of 200 rpm and aeration was performed at a rate of 0.05 vvm. The first strain inoculation concentration was 3 (OD₆₀₀)

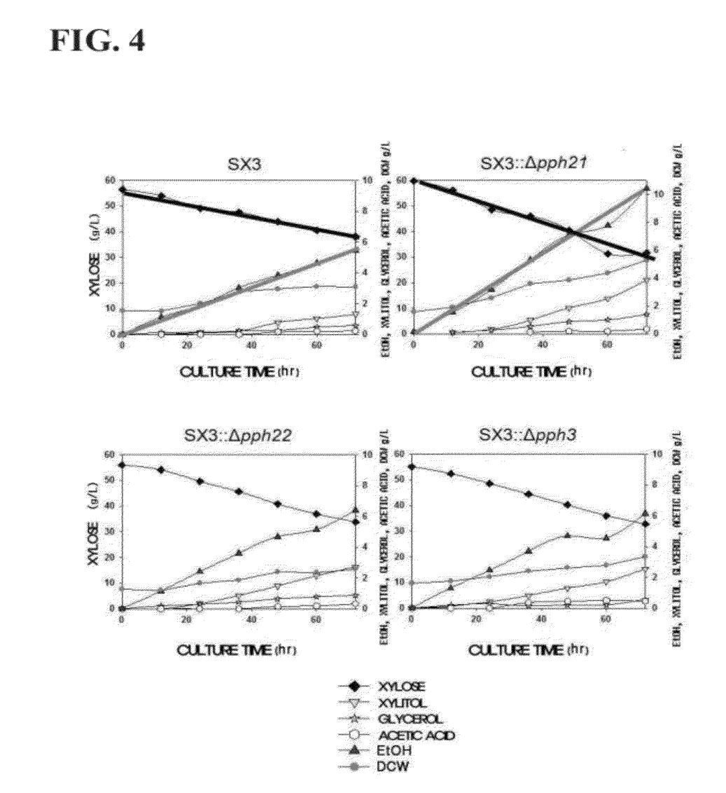

Fermentation results are shown in Table 2 below (See FIG. 4).

TABLE 2

| | Xylose | | Ethanol | | | |
|---|---|---|---|---|---|---|
| | consumption | Final ethanol | production | Yield | | |
| | rate | concentration | efficiency | (g/g (product/xylose)) | | |
| Strains | (g/L · hr) | (g/L) | (g/L · hr) | Ethanol | Xylitol | Glycerol |
| SX3 | 0.25 | 5.45 | 0.08 | 0.30 | 0.07 | 0.03 |
| SX3::Δpph21 | 0.39 | 10.41 | 0.13 | 0.37 | 0.14 | 0.05 |
| SX3::Δpph22 | 0.31 | 6.39 | 0.09 | 0.29 | 0.12 | 0.04 |
| SX3::Δpph3 | 0.31 | 6.14 | 0.09 | 0.27 | 0.11 | 0.03 |

As can be seen from Table 2 above, SX3::Δpph21, SX3::Δpph22 and SX3::Δpph3 strains exhibited superior xylose consumption rate, final ethanol concentration and ethanol production efficiency, as compared to SX3 strains. In particular, as compared to SX strains, SX3::Δpph21 strains exhibited a 1.6-fold increase in xylose consumption rate, a 1.9-fold increase in final ethanol concentration, and a 1.9-fold increase in ethanol production efficiency.

### Example 3

### Fermentation of Ethanol Using SX3, SX3::Δppm1, SX3::Δtor1, SX3::Δtpd3 and SX3::Δmaf1 Strains Among Strains Produced in Example 1

Fermentation of ethanol was performed, as fermentation strains, using SX3, SX3::Δppm1, SX3::Δtor1, SX3::Δtpd3 and SX3::Δmaf1 strains among strains produced in Example 1. Fermentation of ethanol was performed under the same conditions as in Example 2 except the fermentation strains.

The fermentation results are shown in Table 3 below.

TABLE 3

| | Xylose | | Ethanol | | | |
|---|---|---|---|---|---|---|
| | consumption | Final ethanol | production | Yield | | |
| | rate | concentration | efficiency | (g/g (product/xylose)) | | |
| Strains | (g/L · hr) | (g/L) | (g/L · hr) | Ethanol | Xylitol | Glycerol |
| SX3 | 0.25 | 5.45 | 0.08 | 0.30 | 0.07 | 0.03 |
| SX3::Δppm1 | 0.43 | 7.60 | 0.11 | 0.25 | 0.12 | 0.04 |
| SX3::Δtor1 | 0.31 | 6.70 | 0.09 | 0.30 | 0.18 | 0.03 |
| SX3::Δtpd3 | 0.29 | 6.54 | 0.09 | 0.32 | 0.17 | 0.05 |
| SX3::Δmaf1 | 0.40 | 8.19 | 0.11 | 0.29 | 0.14 | 0.04 |

As can be seen from Table 3 above, SX3::Δppm1, SX3::Δtor1, SX3::Δtpd3 and SX3::Δmaf1 strains exhibited superior xylose consumption rate, final ethanol concentration and ethanol production efficiency, as compared to SX3 strains.

### Example 4

### Ethanol Fermentation Using SX3, SX3::Δpph21, SX5::Δald6 and SX3::Δpph21::Δald6 Strains Among Strains Produced in Example 1

Fermentation of ethanol was performed, as fermentation strains, using SX3, SX3::Δpph21, SX5::Δald6 and SX3::Δpph21::Δald6 strains among strains produced in Example 1. Fermentation of ethanol was performed under the same conditions as in Example 2 except the fermentation strains.

The fermentation results are shown in Table 4 below.

TABLE 4

| | Xylose | | Ethanol | | | |
|---|---|---|---|---|---|---|
| | consumption | Final ethanol | production | Yield | | |
| | rate | concentration | efficiency | (g/g (product/xylose)) | | |
| Strains | (g/L · hr) | (g/L) | (g/L · hr) | Ethanol | Xylitol | Glycerol |
| SX3 | 0.25 | 5.45 | 0.08 | 3.08 | 0.30 | 0.07 |
| SX3::Δpph21 | 0.39 | 10.41 | 0.13 | 5.28 | 0.37 | 0.14 |
| SX5::Δald6 | 0.26 | 6.13 | 0.09 | 2.82 | 0.32 | 0.16 |
| SX3::Δpph21::Δald6 | 0.46 | 9.58 | 0.13 | 6.90 | 0.29 | 0.04 |

As can be seen from Table 4 above, SX3::Δpph21::Δald6 strains exhibited superior xylose consumption rate, final ethanol concentration and ethanol production efficiency, as compared to SX3 and SX3::Δpph21 strains. In particular, as compared to SX strains, SX3::Δpph21::Δald6 strains exhibited a 1.84-fold increase in xylose consumption rate, a 1.76-fold increase in final ethanol concentration, and a 1.76-fold increase in ethanol production efficiency.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A method for preparing ethanol from xylose using recombinant Saccharomyces cerevisiae wherein the recombinant Saccharomyces cerevisiae is transformed to express xylose reductase (XR) and xylitol dehydrogenase (XDH) and to over-express xylulokinase (XK),
wherein a gene involved in TOR signal transduction pathways in the recombinant Saccharomyces cerevisiae is partially disrupted or entirely deleted to eliminate functions of the gene.

2. The method according to claim 1, wherein the xylose reductase (XR) utilizes NADH as a cofactor and the xylitol dehydrogenase (XDH) utilizes NAD+ as a cofactor.

3. The method according to claim 1, wherein the gene involved in TOR signal transduction pathways is selected from PPH21, PPH22, PPH3, PPM1, TOR1, TPD3 and MAF1.

4. The method according to claim 1, wherein an acetaldehyde dehydrogenase-encoding gene converting acetaldehyde into acetic acid in the Saccharomyces cerevisiae is partially disrupted or entirely deleted to eliminate functions of the gene.

5. The method according to claim 4, wherein the acetaldehyde dehydrogenase-encoding gene is ALD6.
